(19) United States
(12) Patent Application Publication
(10) Pub. No.: US 2026/0347867 A1
(43) Pub. Date: Nov. 5, 2026

(54) Systems and Methods for In-Plant Indigo Biosynthesis via Fusion Enzyme Constructs

(71) Applicant: Jonathan Keith Windham, Darlington, SC (US)

(72) Inventor: Jonathan Keith Windham, Darlington, SC (US)

(21) Appl. No.: 19/191,381

(22) Filed: Apr. 28, 2025

(51) Int. Cl. C12N 15/82; C12N 9/02; C12N 9/10; C12N 9/42; C12N 9/88

(52) U.S. Cl. CPC C12N 15/825; C12N 9/0061; C12N 9/0073; C12N 9/1051; C12N 9/2445; C12N 9/88; C12N 15/8223; C12N 15/8245; C12Y 110/03002; C12Y 114/13008; C12Y 204/0122; C12Y 302/01021; C12Y 401/99001; C07K 2319/60

(57) ABSTRACT

The present invention provides a genetically engineered fusion enzyme, Indicase, that enables the in planta biosynthesis of indigo pigment within plant fiber cells. Indicase comprises a β-glucosidase domain from Persicaria tinctoria and a laccase domain from Gossypium hirsutum, connected by a flexible peptide linker and directed to the vacuole by a vacuolar targeting signal. When expressed in cotton under the control of a fiber-specific promoter, Indicase catalyzes the hydrolysis of indican and the subsequent oxidation of indoxyl to form indigo pigment inside the vacuole. A complementary tricistronic cassette, Trindican, enables endogenous synthesis of indican from tryptophan in non-indican-producing plants. Together, the Indicase and Trindican constructs comprise the Indicase System, which facilitates sustainable, compartmentalized pigment production in transgenic plants and eliminates the need for external chemical dyeing processes.

### FIELD OF THE INVENTION

The present invention relates to the fields of plant biotechnology, metabolic engineering, and enzymology. More specifically, it pertains to the development of a novel genetically engineered fusion enzyme, referred to as “Indicase,” designed to enable the in planta biosynthesis of indigo pigment within cotton fibers and other engineered plant tissues. This invention introduces a sustainable and precise biosynthetic strategy to replace conventional dyeing processes.

### BACKGROUND OF THE INVENTION

Traditionally, the coloration of cotton textiles involves external dyeing methods that require significant amounts of water, energy, and chemical agents. Indigo, a prominent dye used for denim and other fabrics, is conventionally applied through vat dyeing processes that pose environmental challenges due to the use of hazardous chemicals and the generation of substantial wastewater.

Efforts have been made to produce indigo dye biosynthetically within plants by introducing specific bacterial and plant genes responsible for indigo precursor synthesis. However, these approaches have encountered several challenges:

Gene Expression and Stability: Achieving stable and adequate expression of foreign genes in cotton plants has proven difficult, leading to inconsistent dye production.

Metabolic Pathway Integration: Integrating complex metabolic pathways into cotton plants without disrupting native processes remains a significant hurdle.

Yield and Color Consistency: Ensuring that the in-plant biosynthesis yields sufficient quantities of indigo with consistent coloration has been challenging.

The present invention relates to the formation of a novel fusion enzyme, referred to herein as “Indicase,” to facilitate in planta biosynthesis of indigo within cellulosic material, and more specifically within transgenic cotton (Gossypium hirsutum) fibers, in order to alter the color of the cotton or other cellulosic materials. The novel enzyme described herein biosynthesizes indigo within plant cell vacuoles, thus integrating the indigo molecules as a part of the plant itself, rather than using a dye. The final plant product includes indigo, rather than an indigo-precursor or an indigo-derivative, which is novel in the plant kingdom.

This goal is achieved by integrating two key enzymatic functions into a single fusion protein: 1) β-Glucosidase, which hydrolyzes indican (indoxyl-β D-glucoside) to release indoxyl, and 2) Laccase, which oxidizes indoxyl to form indigo within fiber cells. This enzymatic system ensures controlled, in situ indigo biosynthesis, eliminating the need for external dyeing processes. It should be understood that while the present novel enzyme, Indicase, is designed to be used in connection with cotton, it may also be engineered into a variety of other plant species and, accordingly, is not limited to use with cotton alone.

Laccase is an enzyme (a type of protein that speeds up chemical reactions) that belongs to the oxidase family. Specifically, it's a multi-copper oxidase, meaning it contains multiple copper atoms at its active sites. It's found in various organisms including fungi, bacteria, plants, and insects, but it's most commonly sourced from fungi like Trametes versicolor for industrial applications. Laccase catalyzes the oxidation of a wide range of compounds, particularly phenols and aromatic amines, while simultaneously reducing molecular oxygen to water. In simple terms, it helps turn certain colorless or lightly colored substances into colored compounds by enabling oxidation.

β-Glucosidase (beta-glucosidase) is an enzyme that belongs to the glycoside hydrolase family. Its main role is to break down glycosidic bonds, specifically by cleaving the bond between a glucose molecule and another compound (usually an aromatic or phenolic compound). Essentially, it helps release aglycones (non-sugar parts of glycosides) by removing the sugar part (glucose). In the context of textile dyeing, β-glucosidase is used to release natural dyes from their glycoside forms, making them available for binding to textile fibers.

Heretofore, many different methods and processes have been developed to utilize laccase and β-glucosidase to modify the color of textiles, particularly in combination with indigo. The following documents provide some examples of these developed processes, and each of the following documents is hereby incorporated by reference, in their entireties:

U.S. Pat. No. 8,809,033: Methods for Increasing Hydrolysis of Cellulosic Material in the Presence of Cellobiose Dehydrogenase

The present invention relates to methods for degrading or converting a cellulosic material, methods for producing a fermentation product, and methods of fermenting a cellulosic material with an enzyme composition comprising one or more (several) cellulolytic enzymes, a cellobiose dehydrogenase, and a polypeptide having cellulolytic enhancing activity.

U.S. Pat. No. 9,670,614: Color Modification of Textile

The use of a peroxidase, a source of hydrogen peroxide and a mediator for providing a modified color in the textile is described.

U.S. Pat. No. 10,612,059: Methods and Compositions for the Treatment of Cellulosic Biomass and Products Produced Thereby

A two-step method for activating a cellulosic feedstock is described. The feedstock is subjected to a first high temperature activation step at a temperature greater than 190 °C and a second activation step at a lower temperature under alkali conditions. Also described are methods and compositions for the enzymatic hydrolysis of activated cellulose using one or more cellulase enzymes, a surfactant and polyaspartic acid. Also described are products of the methods.

U.S. Pat. No. 10,718,085: Color Modification of Textile

A method for treating dyed textile, comprises contacting the dyed textile with a pectolytic enzyme, wherein the said method is not applied during the abrasion stage when the dyed textile is a denim fabric.

### US Publication No. 2008/0189871: Laccase Mediators and Methods of Use

Novel laccase mediators, including carboxyamido- and cyano-derivatives of 2.6-diamenthoxyphenol that exhibit improved hydrolytic stability and good bleaching performance. The novel laccase enzymes may be employed in conjunction with the 2.6-dimethoxyphenol derivatives of this invention to provide an improved method for bleaching denim fabrics.

### US Publication No. 2013/0269118: Local Color Modification of Dyed Fabrics Using a Laccase System

The present systems, compositions, and methods relate to local color modification of dyed fabrics using a laccase enzyme system.

### WO 2000/043586: Method of Decolorizin2 a Dyed Material in a Predetermined Pattern

A method of decolorizing a dyed material in a predetermined pattern, which method involves providing a dyed material and ink jet printing a solution of an oxidoreductase onto the dyed material in a predetermined pattern. The present invention further provides a dyed material which has been decolorized in a predetermined pattern by a method of the present invention. The dyed material may be, for example, a dyed textile. Also provided is a method of simultaneously decolorizing and printing on a dyed material in a predetermined pattern. The method involves providing a dyed material and ink jet printing on the dyed material in a predetermined pattern with an ink jet ink which contains an oxidoreductase and one or more dyes which are not significantly decolorize by the oxidoreductase. The oxidoreductase may be, for example, a laccase, peroxidase, or diaphorase. If desired, the dyed material may be heated after printing the solution of an oxidoreductase or an oxidoreductase-containing ink jet ink thereon.

WO 2008/023386: Novel Compositions for Biobleachin2 Coupled with Stone Washing of Indigo Dyed Denims and Process Thereof

Disclosed herein are novel compositions for biobleaching coupled with stone washing of indigo dyed denims comprising a blend of glucose oxidase, catalases and cellulases in the ratio of 1.0:10.0:1.0 along with sugar base, peroxide source and optional adjuvants and a process for the said biobleaching coupled with stone washing wherein the process is carried out at optimized conditions of neutral pH (6.5-7) and a temperature of 55 degrees C. Processing time may vary from 15 minutes to 90 minutes depending on the extent of bleach required for the fabric.

### WO 2010/0754202: Laccases and Methods of Use Thereof at Low Temperature

Laccase enzymes and nucleic acid sequences encoding such laccase enzymes are described. The laccase enzymes may be employed in conjunction with mediators in improved methods for modifying the color of denim fabrics. Low temperature and single-bath textile processing using laccase enzymes are also described.

### WO 2011/025861: Combined Textile Abradin2 and Color Modification

Described are compositions and methods for the enzymatic abrading and color modification of dyed textiles. The compositions and methods permit a textile manufacturer to obtain a wide variety of different textile finishes and colors using exclusively enzymatic methods.

### WO 2012/054485: Local Color Modification of Dyed Fabrics Using a Laccase System

The present systems, compositions, and methods relate to local color modification of dyed fabrics using a laccase enzyme system.

Despite the availability of these prior enzymatic and chemical methods, a significant gap remains in the field of sustainable textile coloration. These conventional systems rely on external application of enzymes or dyes to finished textiles, often requiring chemical mediators, oxidative reagents, high-temperature treatments, or water-intensive processes. Such approaches are limited in their efficiency, scalability, and environmental impact.

Indicase addresses this unmet need by enabling a new paradigm-indigo pigment production that occurs entirely within the growing plant. The fusion enzyme facilitates an in vivo, compartmentalized pathway that allows for the safe and efficient conversion of a stable precursor into indigo, directly inside the cotton fiber vacuole. This not only improves pigment retention and consistency but also eliminates reliance on environmentally hazardous chemical processes. By integrating pigment biosynthesis into plant development itself, Indicase offers superior performance, cost savings, ecological advantages, and compatibility with sustainable agriculture practices that were not previously achievable with external treatments.

### SUMMARY OF THE INVENTION

This invention introduces a specially engineered protein called Indicase that helps plants create indigo pigment directly in their cells. Instead of using traditional chemical dyeing methods, this system allows the plant to make its own pigment safely and efficiently during its natural growth process. Indicase works by combining two key enzymes into one: one enzyme breaks down a stable compound called indican into a reactive intermediate, and the other enzyme turns that intermediate into blue indigo pigment. Everything happens inside a special compartment in the plant cell called the vacuole, which keeps the process controlled and safe for the plant.

Indigo is a pigment derived from plant compounds; it is not produced within living plant cells. Indigo precursors must be extracted from leaves, then undergo a chemical reaction to form indigo. After indigo is formed and refined from the solution, it must then be chemically treated to render a water-soluble version (leuco-indigotin or “indigo white”) suitable for the dyeing of fabrics. After the fabric has taken up the leuco-indigotin, a natural reaction with oxygen must take place in order to revert it back into indigo. This process is time consuming and yields by-products that can harm the environment.

To address these issues, the novel fusion enzyme Indicase was designed to biosynthesize indigo within living plant cells. Genetically transformed plants harboring the Indicase System are capable of biosynthesizing indigo within their cells. For fiber crops such as cotton, this means the production of cotton fibers that already contain indigo and are therefore already blue. Depending on the deepness of blue color desired for the final textile product, the Indicase System either eliminates or greatly reduces the need for the vat dyeing process.

The Indicase System is composed of two separate components that each yield separate products to convert the amino acid tryptophan into indigo in a multi-step process. These components are Indicase and Trindican. Trindican produces three enzymes that complete the first three steps of the conversion process by converting tryptophan into indican. From there, Indicase acts on indican to yield indigo. Use of the Indicase System is not confined to cotton, but can be implemented in other species. Both components of the Indicase System are only required in plants that do not naturally produce indican. In plants where indican is already present, Indicase can act alone to produce indigo in living plant cells.

Specifically, Indicase is made from two enzyme parts joined together: the first part is a β-glucosidase from Polygonum tinctorium, which breaks down indican to produce indoxyl. The second part is a laccase from Gossypium hirsutum (GhLAC1), which oxidizes indoxyl to form the indigo pigment. A flexible linker connects these two enzymes to make sure they fold correctly and work without interfering with each other. The entire fusion enzyme is guided to the vacuole using a vacuolar targeting signal (VTS), and it's only activated in cotton fiber cells thanks to a fiber-specific promoter.

There are many advantages to this approach. First, it eliminates the need for external chemical dyeing, which reduces industrial pollution. Second, it ensures that the pigment is made exactly where it's needed-inside the fiber cells-leading to better pigment retention and durability. Third, by keeping the reactive indoxyl confined to the vacuole, the system avoids damaging other parts of the plant. Finally, using only plant-derived components makes Indicase safe, efficient, and compatible with sustainable agriculture.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments below, serve to explain the principles of the invention.

FIG. 1 is a schematic diagram illustrating the structural organization of the fusion enzyme “Indicase,” comprising an N-terminal vacuolar targeting signal, a β-glucosidase domain, a flexible peptide linker, and a laccase domain.

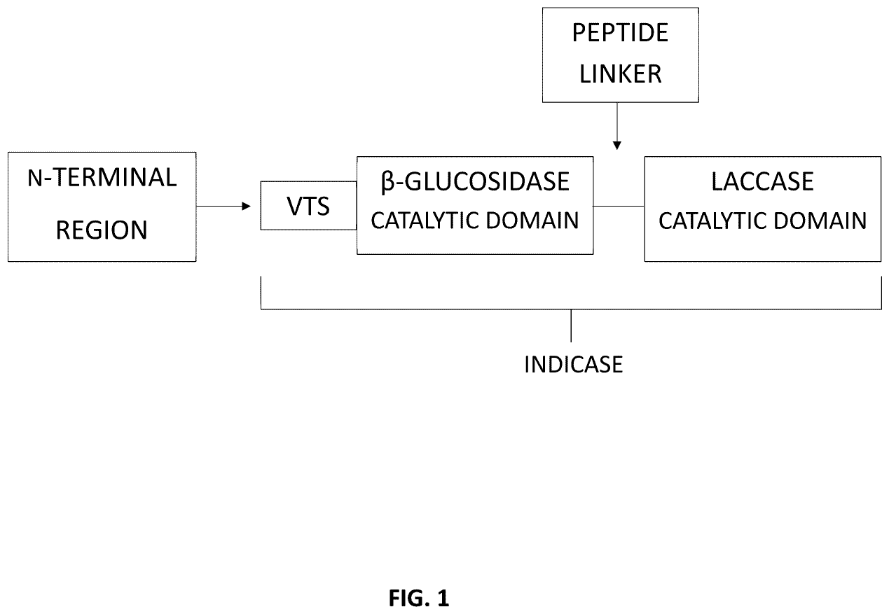

FIG. 2 is a schematic representation of the genetic construct used for plant transformation, showing the arrangement of regulatory elements including a fiber-specific promoter, the Indicase coding sequence, terminator, and associated selection marker.

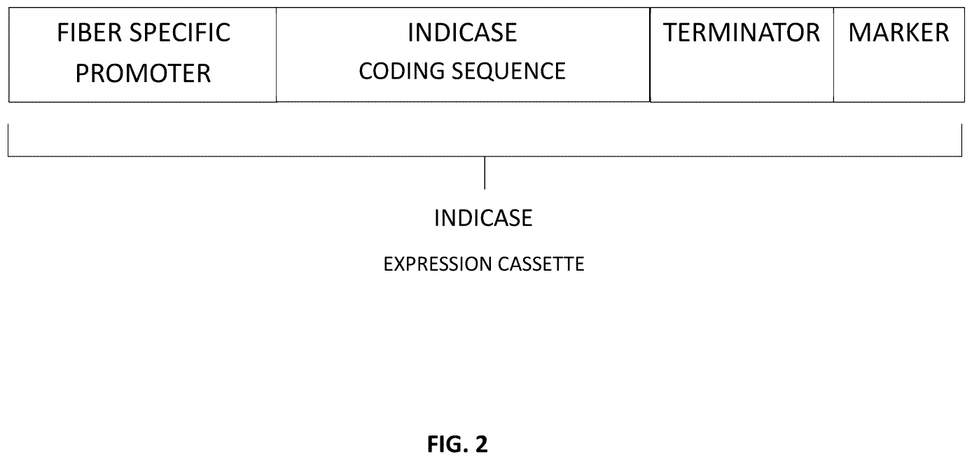

FIG. 3 is a flow diagram illustrating the enzymatic biosynthesis pathway of indigo within plant fiber vacuoles, including the hydrolysis of indican to indoxyl by β-glucosidase and the subsequent oxidation of indoxyl to indigo by laccase.

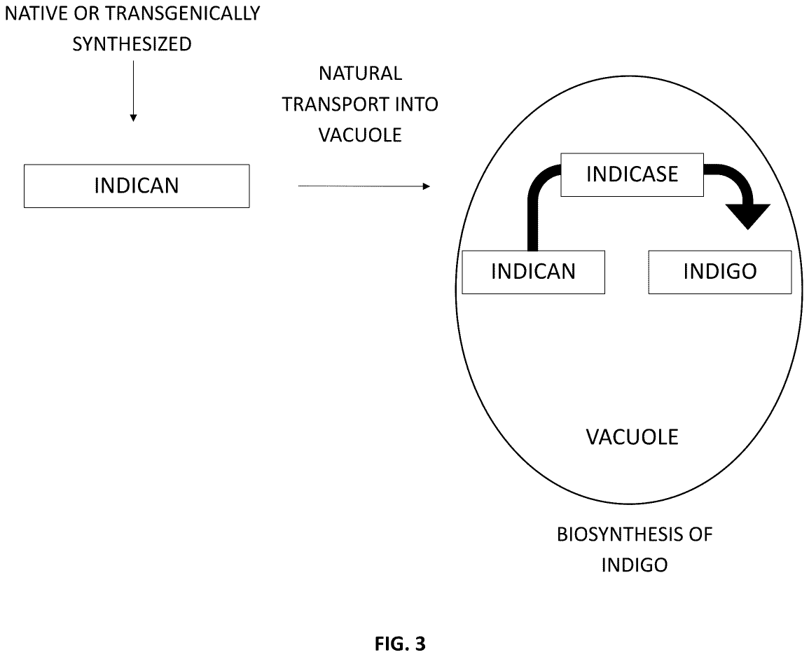

FIG. 4 is a cellular diagram showing the subcellular localization of Indicase within the plant cell, specifically targeting the vacuole, and illustrating in situ pigment accumulation within the fiber cell.

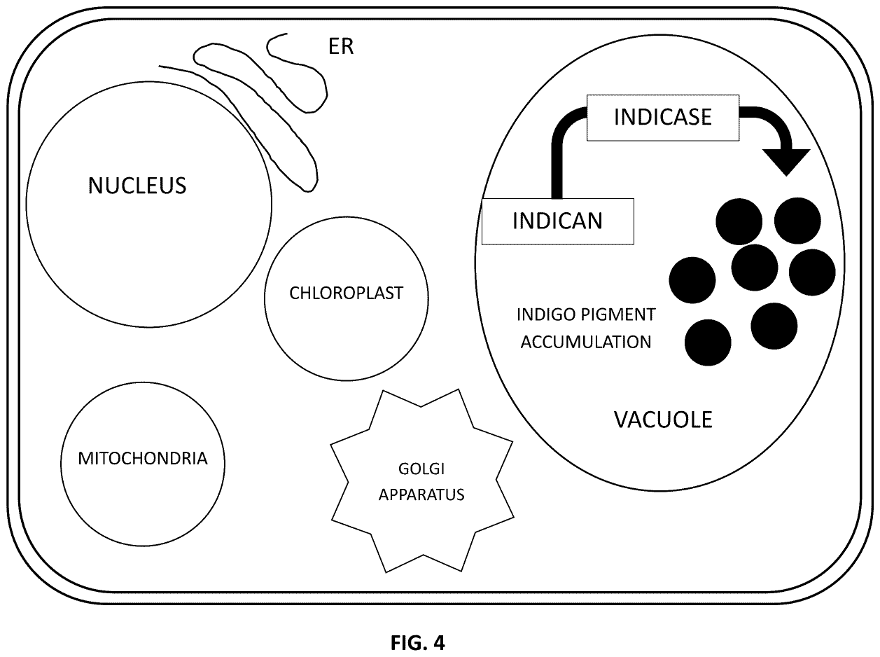

FIG. 5 is a chemical reaction scheme depicting the two-step conversion of indican to indigo, including enzymatic hydrolysis by β-glucosidase and oxidation by laccase in the presence of molecular oxygen.

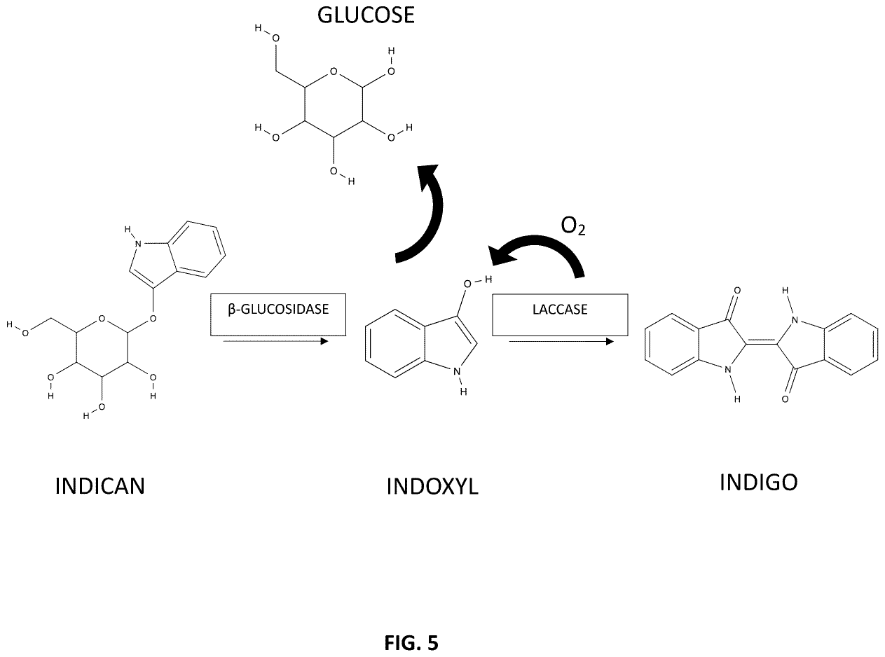

FIG. 6 is a comparative diagram contrasting conventional textile dyeing processes with the genetically engineered in planta biosynthesis of indigo enabled by Indicase in transgenic cotton fibers.

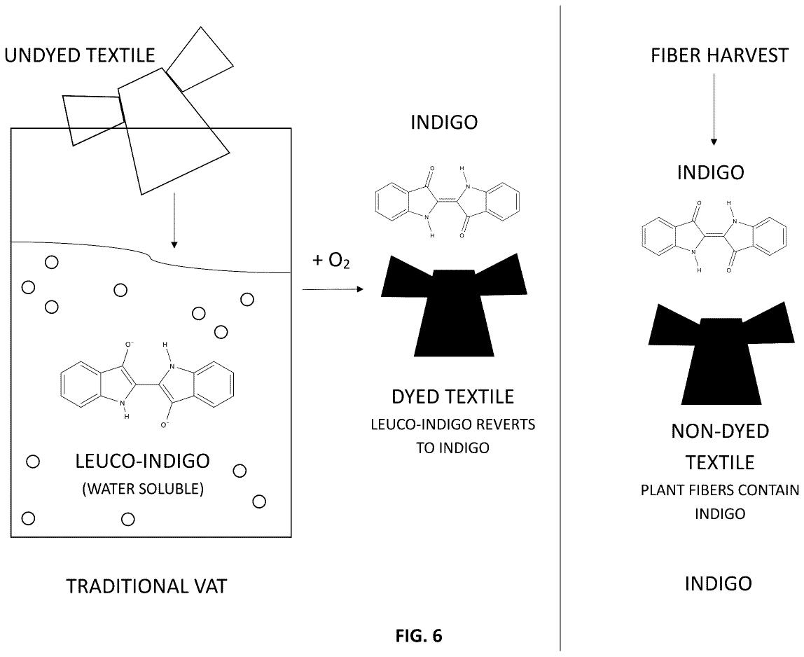

FIG. 7 is a comparative schematic diagram illustrating the biosynthetic pathway enabled by the fusion enzyme Indicase versus prior art methods for indigo dye formation. The diagram shows how, in prior systems, indigo is formed through external application of enzymes or chemical reagents to dyed fabrics, requiring multiple processing steps and chemical inputs. In contrast, the Indicase pathway demonstrates in vivo, vacuole-localized biosynthesis of indigo within the fiber cells of transgenic cotton, enabled by sequential catalytic activity of β-glucosidase and laccase domains within a single fusion protein. This integrated biosynthetic pathway reduces reliance on external processing and chemical dyes.

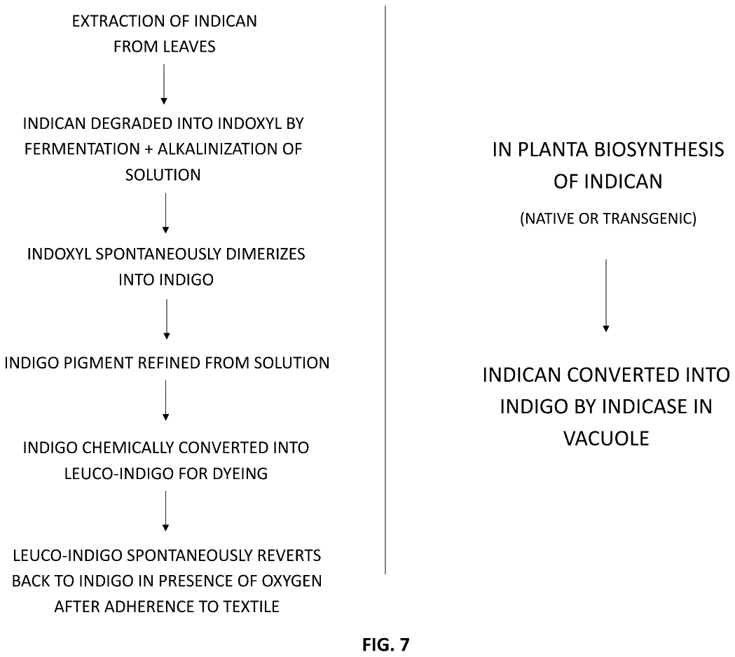

FIG. 8 is a process flow diagram illustrating the stepwise method for the expression and purification of the Indicase fusion enzyme in both microbial and plant systems. The diagram outlines key stages including vector construction, transformation, induction (in microbial systems), protein expression, purification using affinity chromatography, and downstream validation via enzymatic assay and gel electrophoresis. For plant-based systems, the process further includes transformation, transgenic line selection, molecular verification, and subcellular localization analysis. This flowchart enables reproducible production of the enzyme for both experimental validation and application in transgenic cotton.

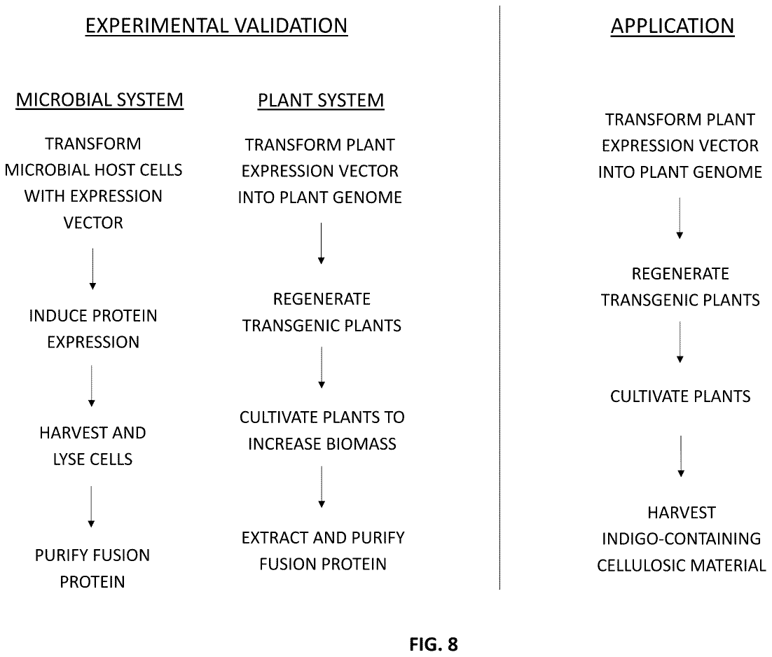

FIG. 9 is a schematic diagram of the structure of a Trindican Cassette.

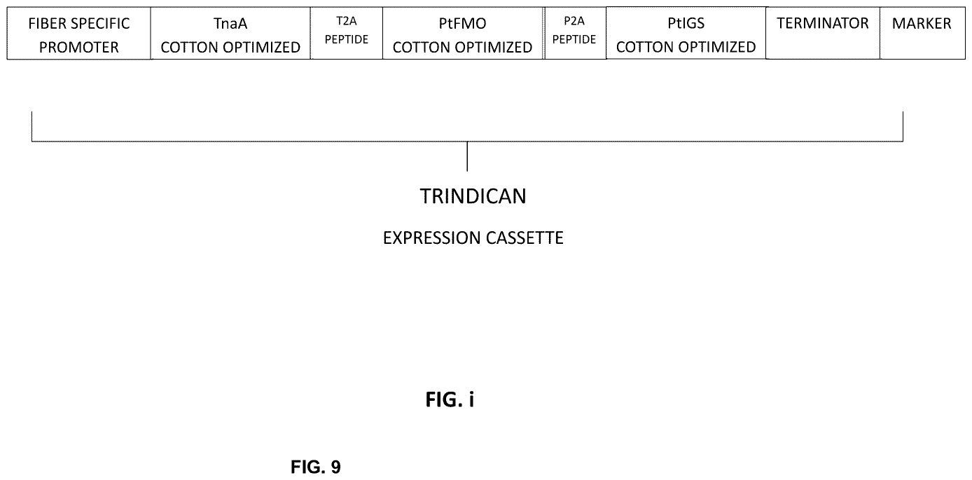

FIG. 10 is an illustration of a step-by-step conversion of tryptophan into indican by the Trindican cassette.

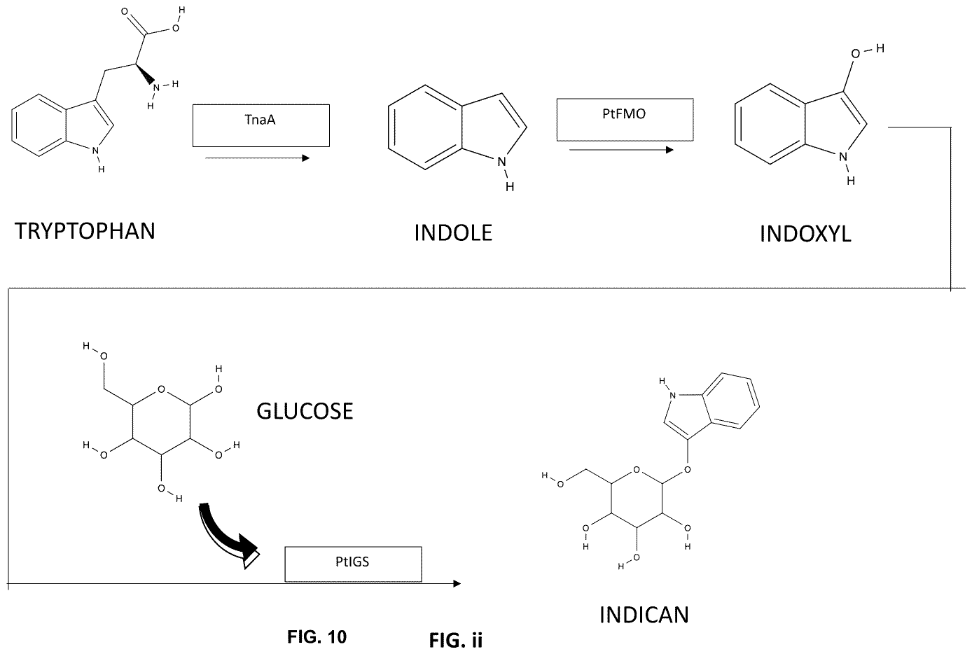

FIG. 11 is a diagram showing indican biosynthesis occurring outside of vacuole. After biosynthesis, indican is naturally stored in the vacuole where Indicase waits to convert it into indigo.

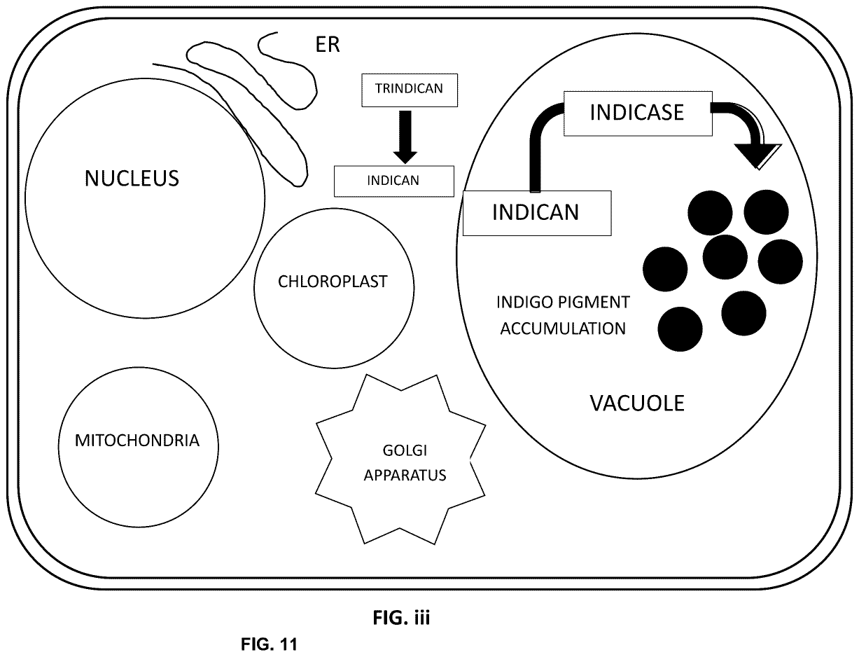

FIG. 12 is a flow chart illustrating steps taken in transgenic plants harboring the Trindican cassette to convert the native amino acid tryptophan into indican.

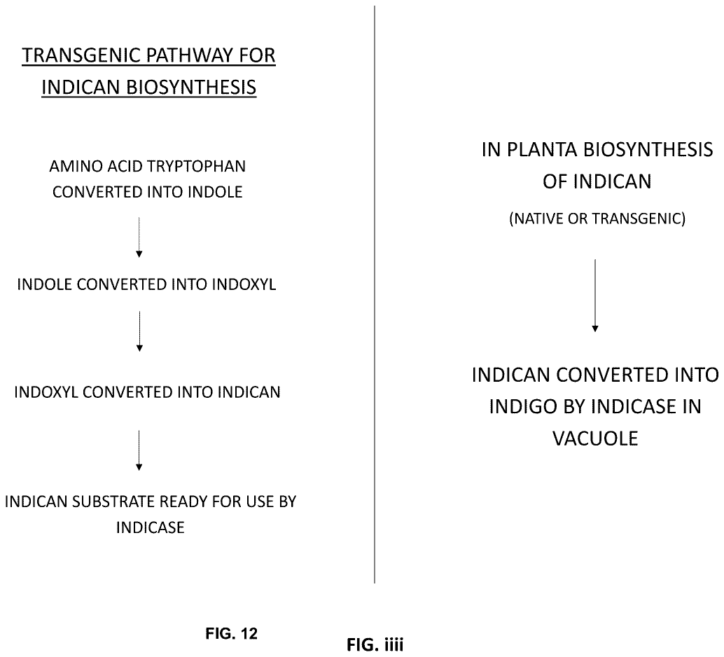

### DETAILED DESCRIPTION OF THE INVENTION

### 1. Technical Overview of the Invention

Indigo is not naturally produced within plant cells, but rather, is the by-product of natural, plant-derived compounds undergoing a natural chemical reaction. These plant-derived compounds must be first extracted and then subjected to the appropriate chemical reactions to yield indigo. Plants that produce these pre-cursor compounds include True indigo (Indigofera tinctoria), Guatemalan indigo (Indigofera suffruticosa), Japanese indigo (Persicaria tinctoria, formerly Polygonum tinctorium), Broad-leaved indigo (Marsdenia tinctoria), and Woad (Isatis tinctoria). Despite a few minor enzymatic differences between species, most of these plants all share a core biosynthesis pathway that yields indican (indoxyl-β-D-glucoside) via glucosylating indoxyl:
Indole-3-Glycerol Phosphate (IGP)→Indole→Indoxyl→Indican (indoxyl-β-D-glucoside)

Indican (indoxyl-β-D-glucoside) is a non-toxic, water-soluble precursor to indigo. It is a stable molecule and stored in the plant vacuole. During fermentation or leaf damage, indican is hydrolyzed enzymatically by a β-glucosidase to yield indoxyl (again) and glucose. Indoxyl then dimerizes spontaneously in the presence of oxygen (O₂) to form indigo (the dimerization of indoxyl is not enzymatically mediated):
Indican (indoxyl-β-D-glucoside)→Indoxyl→Indigo

Indoxyl is highly reactive, unstable, and cytotoxic. It is not an ideal precursor for storage in the plant vacuole. Indican, however, is stable and non-toxic, allowing the plant to safely store it in the vacuole. Some species prefer to enzymatically convert indoxyl into isatan B rather than indican, however the hydrolysis of both molecules yields back indoxyl which then spontaneously dimerizes in the presence of oxygen into indigo. Regardless of which precursor is formed, both are stored in the vacuole and are only degraded into indoxyl through fermentation or physical damage to the leaf. This means that the spontaneous, non-enzymatic dimerization of indoxyl into indigo occurs outside of the living plant cell and relies on the availability of atmospheric oxygen.

Indicase is a novel fusion enzyme that combines a β-glucosidase derived from Persicaria tinctoria fused to a laccase derived from Gossypium hirsutum. The design of Indicase allows for the simultaneous cleavage of indican (indoxyl-β-D-glucoside) into indoxyl+glucose (via the β-glucosidase domain) and then the enzymatic oxidation of indoxyl into indigo (via the laccase domain). Since the oxidation of indoxyl in the Indicase System is driven enzymatically, rather than passively, this means that the formation of indigo does not rely on exposure to atmospheric oxygen which in turn allows for the synthesis of indigo within a living plant cell.

The two catalytic domains of Indicase are fused by a semi-flexible linker peptide that allows for the proper folding of each enzymatic component and lends itself to an improved substrate cascade. This linker and the subsequently improved substrate cascade reduces the risk of off-targeting enzymatic activity of the β-glucosidase or the laccase. Further, Indicase is designed to include a vacuolar targeting signal (VTS) which ensures that its enzymatic activity is confined to the plant cell vacuole. This VTS built into Indicase was derived from the well-characterized CT10 VTS. CT10 usually yields a C-terminus-specific peptide. The Indicase VTS represents a truncated version of CT10, where C-terminus specificity has been removed, allowing the signaling peptide to be attached to the N-terminus in Indicase. This prevents the VTS from interfering with proper protein folding during the assembly of the Indicase enzyme while still allowing the enzyme to be directed to the vacuole for the biosynthesis of indigo to occur there.

Indicase was specifically designed to use indican (indoxyl-β-D-glucoside) as a starting substrate in order to produce indigo. This is significant in that Indicase will only form indigo within plant cells where indican (indoxyl-β-D-glucoside) is already present. In order to extend the usefulness of Indicase to plants that don't naturally produce indican, a complementary cassette containing a carefully designed, full metabolic pathway yielding indican was designed. This cassette is called Trindican.

Trindican was so named because it is a tricistronic expression cassette that expresses three enzymes that convert the amino acid tryptophan into the final product indican. The first of the three enzymes encoded for by the Trindican cassette is TnaA (a tryptophanase) derived from E. coli, with the nucleotide sequence deviating from the wild-type sequence in that it has been codon-optimized for expression in plants. Although the codon-optimized nucleotide sequence differs from the wild-type sequence, the final amino acid sequence (i.e. enzyme) is identical to the wild-type amino acid sequence found in E. coli. TnaA is the first enzyme expressed in this construct as it acts on the initial substrate tryptophan by converting it to indole.

The second enzyme expressed by the Trindican cassette is PtFMO (flavin-containing monooxygenase) derived from Persicaria tinctoria. The nucleotide sequence of PtFMO in the Trindican cassette has been modified from the wild-type sequence in that it is codon-optimized for expression in cotton (Gossypium hirsutum), however, the final amino acid sequence (enzyme) is identical to the wild-type amino acid sequence. This enzyme catalyzes the oxidation of indole into indoxyl. The nucleotide sequence encoding [enzyme] has been codon-optimized for expression in Gossypium hirsutum and is therefore synthetic, non-naturally occurring, and structurally distinct from the wild-type sequence. Although the codon-optimized version encodes the same amino acid sequence as the native enzyme, the nucleotide sequence differs significantly in codon usage, GC content, regulatory compatibility, and mRNA stability, rendering it suitable for expression in plant systems and distinct under 35 U.S.C. § 101.

The third and final enzyme expressed by the Trindican cassette is PtIGS (an indican synthase) also derived from Persicaria tinctoria. The nucleotide sequence of PtIGS in the Trindican cassette has been modified from the wild-type sequence in that it is codon-optimized for expression in cotton (Gossypium hirsutum), however, the final amino acid sequence (enzyme) is identical to the wild-type amino acid sequence. This enzyme glucosylates indoxyl into indican, the precursor needed for Indicase to create indigo.

The enzymes in the tricistronic Trindican expression cassette utilize plant-optimized, self-cleaving peptides (T2A and P2A), which allows for the polycistronic expression of separate enzymes from a single transcript, that is to say that each enzyme is not covalently fused, but they are still expressed from the same open reading frame.

The Indicase cassette and the complementary Trindican cassette together comprise the Indicase System and once transformed into a selected plant genome, work in tandem to produce indigo in the vacuoles of those transgenic plants. Both cassettes harbor a unique genetic marker dubbed “JOB1925”. This marker is a unique, not-naturally-occurring genetic signature used for quick PCR testing of the presence of either cassette in a plant's genome. This marker allows for the rapid and accurate identification of plants that have been transformed with the Indicase System, either legally or illegally. The JOB1925 marker has been carefully designed to be a non-translating, biologically inert, short nucleotide sequence encoded in stop codon-flanked triplets. The marker is placed at the end of each cassette, downstream of the terminator sequence.

Lastly, for specific expression in cotton fibers, each cassette in the Indicase System has been placed under the control of a fiber-specific promoter (GhSCFP) and a plant-compatible terminator (HSP18.2).

The GhSCFP promoter was patented in China (CN 1995350A) in 2006, however, this patent is now listed as expired and its sequence is publicly available (GenBank Accession: GQ411495.1). GhSCFP was chosen because:
1) it is native to the cotton (Gossypium hirsutum) genome, ensuring compatibility within transformed cotton plants.
2) it ensures that expression of the Indicase System is restricted to cotton fiber cells. This prevents the System from producing indigo in off-target tissues such as leaves, petals, roots, etc., and
3) it ensures temporal expression of the Indicase System at the correct developmental stage in cotton fiber development (~18 days post-anthesis (DPA) during secondary wall synthesis with activity peaking 20-25 days DPA) to avoid interference with fiber elongation and to ensure that indigo is biosynthesized before secondary wall cellulose thickening which would hinder the diffusion of indigo throughout the cotton fiber.

The Heat Shock Protein 18.2 (HSP18.2) terminator is derived from Arabidopsis thaliana. It is commonly used in synthetic biology due to its strong and reliable transcriptional termination capability and is well-characterized. HSP18.2 is compatible across a wide range of plant species and its small size (~200 bp) allows it to fit neatly within compact cassettes such as those found in the Indicase System. The HSP18.2 terminator is not patented.

In conclusion, the Indicase cassette and the Trindican cassette together form the Indicase System and work in tandem to biosynthesize indigo within plant vacuoles.

### 2. Structure and Components of Indicase

To understand how Indicase works, it's helpful to look at how its different parts come together like pieces of a machine. Each component of this enzyme plays a specific role in making sure the right chemical reactions happen in the right place inside the plant cell. From start to finish, Indicase is built with instructions that tell it where to go in the cell, how to start the indigo production process, how to keep its parts working together smoothly, and how to complete the final chemical synthesis of indigo pigment. Together, these elements allow the enzyme to operate effectively within the cotton fiber cells, turning a safe and stable compound into the familiar blue pigment—without needing any external dyeing.

The Indicase enzyme is a precisely designed fusion protein composed of several genetically engineered components that, once transcribed and translated in vivo from the Indicase cassette, work synergistically to perform its indigo-producing function within plant fiber cells. Transcription of the Indicase cassette is placed under the control of a fiber-specific promoter, GhSCFP. This promoter is native to the cotton (Gossypium hirsutum) genome and serves to initiate assembly of Indicase only in cotton fiber cells and at the correct developmental stage. The protein assembly begins with the inclusion of a Vacuolar Targeting Signal (VTS) derived from a truncated CT10 peptide. This peptide is placed at the N-terminus of the enzyme to guide the entire protein to the vacuole of the plant cell, ensuring that enzymatic activity is restricted to the correct subcellular compartment.

Next, the β-glucosidase gene incorporated into the Indicase coding sequence and derived from Polygonum tinctorium yields a functional β-glucosidase enzyme. To adapt this enzyme for use in the vacuole, its native chloroplast transit peptide (cTP) is deleted, as it is unnecessary and would otherwise misdirect the enzyme to the chloroplast. This β-glucosidase domain is responsible for hydrolyzing indican, a glucoside precursor, to release indoxyl within the vacuole.

Connecting the β-glucosidase to the next functional component is a flexible peptide linker. This linker is composed of repeated GGGGS motifs, known for their flexibility, and an EAAAK motif, which provides limited rigidity and spacing. This hybrid structure maintains the physical separation of the catalytic domains and supports proper folding and independent function of each domain.

Following the linker nucleotide sequence is the GhLAC1 gene, a laccase from Gossypium hirsutum, included in its full-length native form. The laccase domain encoded by this sequence catalyzes the oxidation of the indoxyl intermediate into indigo. By maintaining the laccase sequence in its unmodified state, native activity and compatibility within cotton fibers are preserved.

The HSP18.2 terminator derived from Arabidopsis thaliana is appended to the end of the nucleotide sequence to properly terminate translation. Finaly, downstream of the terminator resides the JOB1925 selectable marker. This artificial marker is biologically inert and does not yield any functional protein product, nor does it affect the structure of Indicase. Its placement at the very end of the Indicase nucleotide sequence serves to allow for the tracking of the presence or absence of the Indicase sequence within any tested plant material via a rapid PCR test. This composite genetic structure allows Indicase to function as a single, multifunctional enzyme capable of carrying out the full conversion of indican to indigo within the vacuole of cotton fiber cells.

### 3. Enzyme Function and Mechanism of Action

The enzymatic function of Indicase can be understood as a simple two-step process that helps the plant make indigo pigment directly in its fibers. First, Indicase breaks down a stable compound called indican, which is safely stored in plant cells, into a more reactive substance called indoxyl. Then, this indoxyl is quickly turned into indigo, the familiar blue pigment, all within a protected part of the cell called the vacuole. By keeping everything inside the vacuole, the plant avoids damage from the reactive indoxyl and ensures the pigment stays exactly where it's needed—inside the cotton fiber itself.

The functionality of Indicase relies on a carefully coordinated two-step enzymatic pathway, designed to transform the precursor molecule indican into the final pigment indigo within the vacuoles of plant fiber cells. The first step of the reaction is carried out by the 0-glucosidase domain of Indicase, which hydrolyzes the glycosidic bond in the substrate indican (indoxyl-β-D-glucoside). This enzymatic cleavage releases two products: a sugar molecule (glucose) and the aglycone compound indoxyl. The hydrolysis occurs within the vacuole, a controlled and enclosed subcellular compartment, ensuring the released indoxyl does not diffuse into other parts of the cell where it could cause oxidative damage.

Immediately following the hydrolysis, the second step of the reaction is catalyzed by the laccase domain (GhLAC1) of Indicase. The laccase domain of Indicase catalyzes the oxidation of indoxyl to promote its efficient dimerization into insoluble indigo within the vacuole, minimizing reliance on spontaneous oxidation and enhancing spatial control of pigment formation. The laccase enhances the rate of oxidation and ensures that the process is efficient and complete, even under the low-oxygen conditions often found in plant vacuoles. By coupling these two enzymatic steps within the same protein and within the same compartment, Indicase ensures substrate channeling: the rapid and direct transfer of indoxyl from the β-glucosidase to the laccase active site.

Although GhLAC1 is a naturally occurring laccase in Gossypium hirsutum, its incorporation into the Indicase fusion enzyme in tandem with a β-glucosidase domain, a synthetic flexible linker, and a vacuolar targeting signal creates a novel, non-obvious structure not found in nature. This engineered configuration enables in vivo substrate channeling and compartmentalized indigo biosynthesis within fiber cell vacuoles-capabilities not inherent to GhLAC1 alone.

The indigo pigment, once formed, precipitates in the aqueous environment of the vacuole due to its low solubility. This precipitation anchors the pigment within the cotton fiber, leading to uniform pigmentation and enhancing the durability of the pigment. Because this transformation occurs intracellularly, it obviates the need for post-harvest chemical dyeing and minimizes environmental contamination, representing a novel, efficient, and eco-friendly approach to plant-based pigment production.

### 4. Molecular Cloning and Construct Assembly

This section explains how the pieces of DNA needed to build Indicase are put together and prepared for use in cotton plants. Think of this process like writing a recipe: each part of the enzyme—the instructions for its tools and where they should go—is carefully selected, trimmed, and stitched together in the right order. Once all the parts are combined into one complete genetic sequence, it can be inserted into living cells for testing and use. This step is the foundation that allows Indicase to be made inside cotton plants and do its job.

To construct the Indicase fusion enzyme, the process begins with gene sourcing and primer design. The cDNA sequences for the β-glucosidase gene from Polygonum tinctorium and the laccase gene (GhLAC1) from Gossypium hirsutum are obtained from publicly available genetic databases. Specific primers are designed to amplify these sequences, incorporating restriction sites for XbaI and KpnI at the 5′ and 3′ ends respectively, to ensure proper directional cloning.

Following gene amplification, the β-glucosidase gene undergoes sequence optimization. This includes the removal of its native chloroplast transit peptide (cTP), which is not needed in the vacuolar targeting context, and codon optimization to improve expression efficiency in cotton plants. In parallel, a synthetic peptide linker composed of the repeating sequence GGGGS flanking a central EAAAK segment is synthesized. This linker ensures the structural flexibility of the fusion protein while maintaining the proper orientation and activity of each domain. Additionally, an N-terminal vacuolar targeting signal (VTS) derived from a truncated version of the CT10 peptide is included upstream of the β-glucosidase coding sequence to direct the full fusion enzyme to the vacuole.

The complete gene construct is assembled in a plant-compatible expression vector, such as pCAMBIA1301, which supports plant transformation and includes necessary selection markers. The final linear arrangement of the construct is: Fiber Specific Promoter—ATG (start codon)—VTS—β-glucosidase—Linker—GhLAC1—Terminator—JOB1925 Marker.

An assembled plasmid compatible with microbial systems is then introduced into competent E. coli cells via transformation for amplification and validation. Selected bacterial colonies are screened for successful incorporation of the full-length Indicase construct through colony PCR and confirmed by DNA sequencing to ensure sequence fidelity and correct orientation before proceeding to plant transformation steps.

### 5. Expression and Purification

Once the Indicase gene is assembled, it needs to be produced and checked to make sure it works. This part of the process is about making the enzyme inside cells and then separating it out so it can be tested. In simple terms, scientists give the genetic instructions to either bacteria or plant cells, and those cells start making Indicase. After enough enzyme is made, it's collected, cleaned up, and tested to confirm that it functions the way it should. This ensures that everything is working before using Indicase in a real cotton plant.

Expression and purification of the Indicase enzyme can be conducted in both microbial and plant systems depending on the purpose of study and final application. In microbial systems, the Indicase construct is cloned into an expression vector compatible with IPTG-inducible E. coli strains. After transformation, colonies are cultured in appropriate media, and protein expression is induced by the addition of IPTG once the culture reaches the desired optical density. Cells are harvested by centrifugation, lysed using mechanical or chemical methods, and the fusion protein is purified using affinity chromatography, typically with a His-tag column. Following purification, protein expression is validated by SDS-PAGE to confirm size and purity, and enzymatic assays using indican substrate are performed to verify the functional activity of the enzyme.

For plant-based expression utilizing Agrobacterium-mediated transformation, the Indicase gene is cloned into a binary plant expression vector. The construct is introduced into plants via Agrobacterium tumefaciens-mediated transformation. Transgenic lines are selected using antibiotic resistance markers or reporter genes. Successful transformants are propagated and screened for Indicase expression using reverse transcription quantitative PCR (RT-qPCR) to confirm transcript presence and Western blotting to confirm protein accumulation. To verify proper localization of the fusion protein to the vacuole, a fluorescent tag such as GFP may be fused to the enzyme and visualized using confocal microscopy. This approach ensures that the enzyme is both functionally expressed and accurately localized within the cotton fiber cells, enabling effective in situ indigo biosynthesis.

### 6. Application in Transgenic Cotton

Now that Indicase has been created and tested, this step shows how it's used in real cotton plants. The idea is to get the plant to make the enzyme in just the right place and at the right time—inside the growing fiber cells, while the cotton is developing. This way, the plant naturally makes the blue indigo pigment during its normal growth process, saving time, cost, and reducing environmental harm. This section explains how scientists guide the plant to use Indicase to make pigment safely and efficiently inside the cotton itself.

During cotton fiber development (18-30 days post-anthesis), Indicase is expressed and targeted to vacuoles. In the presence of indican, either synthesized by the plant or introduced via upstream biosynthetic engineering, Indicase catalyzes its conversion to indigo.

The in-situ pigment becomes physically integrated within the cotton fiber matrix, eliminating the need for external dyeing. Indigo accumulation is monitored by visual inspection and confirmed by analytical techniques including HPLC, LC-MS, and UV-Vis spectroscopy.

### 7. Full Gene Sequence for Indicase:

ATGCCCCTGTCTTCTATTCTGCGTAACATAAGCATTCCGCTTAAGCGGAC
TAGTTTCCCCAAGAAATTCCTGTTTGGGGCTGGCTCTGCTTCTTACCAAT
ATGAAGGAGCAGCACATATAGATGGGCGAGGACTTAGCGTCTGGGATGTC
TTCACTAAGGAACACCCTGAAAAGATCGCAGATCAGTCGAATGGAGATGT
TGCTCAAGACTTTTATCACCGATACAAGGAAGATATAAAGTCGATGAAGG
AAATGGGTTTGGAGTCATTCAGGTTCTCCATTTCATGGTCAAGAATATTA
CCTAATGGGAAAATCAGTGGAGGAATCAACAAGCTTGGGATCAAGTTCTA
CAATAATCTCATTGACGAACTGCTAGCCAATGGAATCAAGCCACTTGTCA
CTATCTACCATTGGGACCTTCCACAAGCACTTCAAGACGAATATGGAGGG
TTCTTGAGCCCCAAAATCGTGGATGACTTTCTGGAATATGCAAACCTAGT
TTTTAAGGAGTTCGGGGATAGGGTTAAGCATTGGGCGACACTGAATGAAC
CCAATATAATGACCCAACAAGGGTACGTATTTGGGGCACATGCACCCGGA
CGATGTTCTCACTTCGAATGGAACTGCCCGGCTGGAAACTCCGGCACCGA
GCCTTATATAGTTGGTCACCACCTCCTCCTATGTCATGCTGCAGCTTTTC
AACTATACAAACAAAAGTATAAGGATGATCAAAAGGGTATAATCGGAATA
ACAACCGCGACACAGATGGCCATACCGTTAAACGACAACGTTGCCAACCT
CTTGGCAGCGTCACGAGCCATCGATTTCAACATTGGATGGTTTTTGCATC
CGGTTGTTTACGGCGAGTATCCACAGACGATGAGGGAGCGGTTGGGAAGT
CGACTGCCAAAATTCACAGAAAAAGAGTCGGAGATGTTGAAACAATCGTT
CGACTTTATAGGGTTGAATTACTACTCAACTGATTATGCAGCCGCATCAT
CTTTTTCAGTTGATCCAGTGAATGTCAGTTACACAACTGATTCCCGAGCA
ACATTATCAGCGATAAAAGATGGGGTTCCTATCGGCGACCCGACATTTAT
GAGCTGGTTGCATATATATCCAGAGGGCATCCTAACTCTGTTGCGATACG
TAAAGGAAAGGTACAACAATCCATTTGTCATGATCACTGAGAATGGGATG
GCCGATGAAAACAAGGGATCATTAGCGGAAGATCCGATGGCTTTAAAAGA
CAACGTCAGAATTCGATATCACCGCGAACATCTATACTATGTTCTTGAAG
CTATAAAGGAGGGTGTGAACGTGGGAGGATACTACGCATGGACATGGATG
GATGATTTCGAGTGGGGTTCTGGATATACTCCTCGATTCGGTCTCAACTT
TGTGGATTTCGACAATGATTTGAAGAGAACCCCCAAGGATTCTTACTTCT
GGTTCAAGGACTTCCTTGCAAATGGTGGTGGTGGTTCTGGTGGTGGTGGT
TCTGAGGCTGCTGCTAAGGGTGGTGGTGGTTCTGGTGGTGGTGGTTCTAT
GGGTTTACAGCAAGGTTTAGTGACATGGTTTGTAGGGGTTCTATTTCTAA
GCACTTTGCTCCTTTCCAATGCAGATGTACATCACTATGAATTCTTTGTG
CGAGAGTCAAATTTTACTAAGCTGTGCAACACAACGACATTGCTGGTCGT
AAACGACAGCTATCCAGGGCCTGAGATTCGGGTTCACAGAGGTGATACTG
TCTTCGTTAATGTCCACAATCAAGGAAACTACGGCTTCACCATTCACTGG
CACGGCGTGAAACAACCGAGGAATCCATGGTCCGACGGTCCCGAGTTCGT
AACGCAGTGCCCCATCCAACCAGGGACCAACTTCACCTATGAAATCGTAC
TATCGGATGAAATAGGAACACTGTGGTGGCACGCACACAGTGACTGGACT
CGCGGTTCCGTCCATGGAGCCTTCGTCATTTTGCCGGCCAAGAAAGAAAC
TTATCCGTTCCCCACGCCAGACGCCGATCAAACTATCATACTTGAATCAT
GGTACGATGGGGACTACAAGCAAATTATTGATGACGCACTTGCCGCCGGT
GCCTCTCCTCGCCAACCAAGTGCTTATGCTATCAGTGGACACGTCGGAGA
CACATATGGATGCCCCAATGATACAATATTCCGTATGCAAGTTGATTCTG
AGAAGATATACCTTCTCCGAATAATTAACGCTGCAATGAACGAACATTTT
TTCTTCACCATCGCGAACCACACCCTCACAGTCGCCGCACAAGATGCCTC
CTATGTTCGAAGGTTTACGAGGGACTATATATTGATAAGCCCTGGCCAAA
CCATGGATGTTTTAGTCTCTGCCAATCGAAACGTTGGCCAATATTACATG
GCTATTAGGCCTTTCTCTGATTCCTCTGCTGCACCTGTTGACAACATCAC
TACTGGCATTTTTGAATATACAAACAGTGAGGGCGGATTGAATGCTTCCT
TGATAACGTTGCCCGTAATGAACGATACAGATGCTATGATTAACTTCCTA
AACCAAATTCGGAACACGAAAGTCTCCCAGAATCCGCGGATAAATGTGCC
GGCAGATAAGGATATTAAAAGACGAGTGTTCATGACACTCGCGGTAAACA
ACTTGCCTTGCAACACCTGTGTTGTAGGCAGCAGACTTGTTGCAAGTTTG
AACAATGTAAGCTATGTTTCCCCAAGTATTGACATTCTCCAAGCATACTA
CAACAGGAACATAAGTGGTGTTTACACCGAAGATTTTCCATTGAATCCCC
CAGTAATCTATGATTTCACTGGAAACTTGACTAATCTCAATACCCCTGTT
GAGGAAGGGACGAGGGTAATAGTGGTAAATTATGGGGAAGGAGTTGAGAT
GGTGCTGCAAGCAACCCAGATGGGGGCTGGTGGAAGTCACCCAATCCATT
TGCACGGTTCTAGCTTCTATTGGGTGGGAACAGGTTTCGGAAATTTCAAC
AATAAGACAGACCCAAGGACTTATAATCTGGTTGATCCACCACTCATTAA
CACTGTCCATGTTCCTGGTAGAAGATGGGTTGCCATCAGATTTTTTGCTA
CCAATCCCGGAGTATGGTTTATGCATTGCCATTTGGAGAGGCATAGTAGC
TGGGGAATGGACACTGTTCTCATCGTGAGGAATGGTAAAACCAAGAAAAC
AAGCATCCGCCCACCGCCATCTACCATGCCTCGTTGTCCTGGAACCTAG

### 8. Full Protein Sequence for Indicase:

MPLSSILRNISIPLKRTSFPKKFLFGAGSASYQYEGAAHIDGRGLSVWDV
FTKEHPEKIADQSNGDVAQDFYHRYKEDIKSMKEMGLESFRFSISWSRIL
PNGKISGGINKLGIKFYNNLIDELLANGIKPLVTIYHWDLPQALQDEYGG
FLSPKIVDDFLEYANLVFKEFGDRVKHWATLNEPNIMTQQGYVFGAHAPG
RCSHFEWNCPAGNSGTEPYIVGHHLLLCHAAAFQLYKQKYKDDQKGIIGI
TTATQMAIPLNDNVANLLAASRAIDFNIGWFLHPVVYGEYPQTMRERLGS
RLPKFTEKESEMLKQSFDFIGLNYYSTDYAAASSFSVDPVNVSYTTDSRA
TLSAIKDGVPIGDPTFMSWLHIYPEGILTLLRYVKERYNNPFVMITENGM
ADENKGSLAEDPMALKDNVRIRYHREHLYYVLEAIKEGVNVGGYYAWTWM
DDFEWGSGYTPRFGLNFVDFDNDLKRTPKDSYFWFKDFLANGGGGSGGGG
SEAAAKGGGGSGGGGSMGLQQGLVTWFVGVLFLSTLLLSNADVHHYEFFV
RESNFTKLCNTTTLLVVNDSYPGPEIRVHRGDTVFVNVHNQGNYGFTIHW
HGVKQPRNPWSDGPEFVTQCPIQPGTNFTYEIVLSDEIGTLWWHAHSDWT
RGSVHGAFVILPAKKETYPFPTPDADQTIILESWYDGDYKQIIDDALAAG
ASPRQPSAYAISGHVGDTYGCPNDTIFRMQVDSEKIYLLRIINAAMNEHF
FFTIANHTLTVAAQDASYVRRFTRDYILISPGQTMDVLVSANRNVGQYYM
AIRPFSDSSAAPVDNITTGIFEYTNSEGGLNASLITLPVMNDTDAMINFL
NQIRNTKVSQNPRINVPADKDIKRRVFMTLAVNNLPCNTCVVGSRLVASL
NNVSYVSPSIDILQAYYNRNISGVYTEDFPLNPPVIYDFTGNLTNLNTPV
EEGTRVIVVNYGEGVEMVLQATQMGAGGSHPIHLHGSSFYWVGTGFGNFN
NKTDPRTYNLVDPPLINTVHVPGRRWVAIRFFATNPGVWFMHCHLERHSS
WGMDTVLIVRNGKTKKTSIRPPPSTMPRCPGT*

### 9. Full Gene Sequence for the Indicase Cassette:

ACAACTTTTCTCTACCAATCAAATTTAAAAAATAGAAAAATGAAAATCGA
TGAATTGGATCACCACAATTTAGCCCAAAGAAAAACACAGTCAACCCCTC
TCACAGGGTAGGAATGATTTCGAGGTATAGATAGACATAGTAACGGGCAA
CTTTAACTATTGCTGCCTCGATTTGAGGAAAATATCAAATCCAAGACAAA
AATTTCAATTATACACTATGCCATACCATTATAAATATCCCCGTTCGCAA
TATCATCACCATTATTTGAATTTGCATTGCAACATTCGTCACCGTTAGTT
ATACCATCACCATCACTTAATTACTAAAATAATTATTGGTTTCTCAATAT
GAAAAAGCTCGAGTGCATTTTCTTTTGAATATCAACCGAAAAGAAAGGAA
AAACTAAAGATTTTGGAAGGTGACGGGGAAACCAAAAAGGAAATTTTGGG
CATTTTTAAAATGAGAAAGACGAATGTAATAACCCATTTTTCTTTCTTAC
TCTGACAACGCCACAGATGCTTTACATGCATCATGTGATCGTGGGGGACC
CGAAACTTGGCATACGGAAAGCACCAACGGCACAGCATTAAAAGAAATTG
TGTATAATGTTAAAAGACCATTAATTCAGTCTCATGCCCCTGTCTTCTAT
TCTGCGTAACATAAGCATTCCGCTTAAGCGGACTAGTTTCCCCAAGAAAT
TCCTGTTTGGGGCTGGCTCTGCTTCTTACCAATATGAAGGAGCAGCACAT
ATAGATGGGCGAGGACTTAGCGTCTGGGATGTCTTCACTAAGGAACACCC
TGAAAAGATCGCAGATCAGTCGAATGGAGATGTTGCTCAAGACTTTTATC
ACCGATACAAGGAAGATATAAAGTCGATGAAGGAAATGGGTTTGGAGTCA
TTCAGGTTCTCCATTTCATGGTCAAGAATATTACCTAATGGGAAAATCAG
TGGAGGAATCAACAAGCTTGGGATCAAGTTCTACAATAATCTCATTGACG
AACTGCTAGCCAATGGAATCAAGCCACTTGTCACTATCTACCATTGGGAC
CTTCCACAAGCACTTCAAGACGAATATGGAGGGTTCTTGAGCCCCAAAAT
CGTGGATGACTTTCTGGAATATGCAAACCTAGTTTTTAAGGAGTTCGGGG
ATAGGGTTAAGCATTGGGCGACACTGAATGAACCCAATATAATGACCCAA
CAAGGGTACGTATTTGGGGCACATGCACCCGGACGATGTTCTCACTTCGA
ATGGAACTGCCCGGCTGGAAACTCCGGCACCGAGCCTTATATAGTTGGTC
ACCACCTCCTCCTATGTCATGCTGCAGCTTTTCAACTATACAAACAAAAG
TATAAGGATGATCAAAAGGGTATAATCGGAATAACAACCGCGACACAGAT
GGCCATACCGTTAAACGACAACGTTGCCAACCTCTTGGCAGCGTCACGAG
CCATCGATTTCAACATTGGATGGTTTTTGCATCCGGTTGTTTACGGCGAG
TATCCACAGACGATGAGGGAGCGGTTGGGAAGTCGACTGCCAAAATTCAC
AGAAAAAGAGTCGGAGATGTTGAAACAATCGTTCGACTTTATAGGGTTGA
ATTACTACTCAACTGATTATGCAGCCGCATCATCTTTTTCAGTTGATCCA
GTGAATGTCAGTTACACAACTGATTCCCGAGCAACATTATCAGCGATAAA
AGATGGGGTTCCTATCGGCGACCCGACATTTATGAGCTGGTTGCATATAT
ATCCAGAGGGCATCCTAACTCTGTTGCGATACGTAAAGGAAAGGTACAAC
AATCCATTTGTCATGATCACTGAGAATGGGATGGCCGATGAAAACAAGGG
ATCATTAGCGGAAGATCCGATGGCTTTAAAAGACAACGTCAGAATTCGAT
ATCACCGCGAACATCTATACTATGTTCTTGAAGCTATAAAGGAGGGTGTG
AACGTGGGAGGATACTACGCATGGACATGGATGGATGATTTCGAGTGGGG
TTCTGGATATACTCCTCGATTCGGTCTCAACTTTGTGGATTTCGACAATG
ATTTGAAGAGAACCCCCAAGGATTCTTACTTCTGGTTCAAGGACTTCCTT
GCAAATGGTGGTGGTGGTTCTGGTGGTGGTGGTTCTGAGGCTGCTGCTAA
GGGTGGTGGTGGTTCTGGTGGTGGTGGTTCTATGGGTTTACAGCAAGGTT
TAGTGACATGGTTTGTAGGGGTTCTATTTCTAAGCACTTTGCTCCTTTCC
AATGCAGATGTACATCACTATGAATTCTTTGTGCGAGAGTCAAATTTTAC
TAAGCTGTGCAACACAACGACATTGCTGGTCGTAAACGACAGCTATCCAG
GGCCTGAGATTCGGGTTCACAGAGGTGATACTGTCTTCGTTAATGTCCAC
AATCAAGGAAACTACGGCTTCACCATTCACTGGCACGGCGTGAAACAACC
GAGGAATCCATGGTCCGACGGTCCCGAGTTCGTAACGCAGTGCCCCATCC
AACCAGGGACCAACTTCACCTATGAAATCGTACTATCGGATGAAATAGGA
ACACTGTGGTGGCACGCACACAGTGACTGGACTCGCGGTTCCGTCCATGG
AGCCTTCGTCATTTTGCCGGCCAAGAAAGAAACTTATCCGTTCCCCACGC
CAGACGCCGATCAAACTATCATACTTGAATCATGGTACGATGGGGACTAC
AAGCAAATTATTGATGACGCACTTGCCGCCGGTGCCTCTCCTCGCCAACC
AAGTGCTTATGCTATCAGTGGACACGTCGGAGACACATATGGATGCCCCA
ATGATACAATATTCCGTATGCAAGTTGATTCTGAGAAGATATACCTTCTC
CGAATAATTAACGCTGCAATGAACGAACATTTTTTCTTCACCATCGCGAA
CCACACCCTCACAGTCGCCGCACAAGATGCCTCCTATGTTCGAAGGTTTA
CGAGGGACTATATATTGATAAGCCCTGGCCAAACCATGGATGTTTTAGTC
TCTGCCAATCGAAACGTTGGCCAATATTACATGGCTATTAGGCCTTTCTC
TGATTCCTCTGCTGCACCTGTTGACAACATCACTACTGGCATTTTTGAAT
ATACAAACAGTGAGGGCGGATTGAATGCTTCCTTGATAACGTTGCCCGTA
ATGAACGATACAGATGCTATGATTAACTTCCTAAACCAAATTCGGAACAC
GAAAGTCTCCCAGAATCCGCGGATAAATGTGCCGGCAGATAAGGATATTA
AAAGACGAGTGTTCATGACACTCGCGGTAAACAACTTGCCTTGCAACACC
TGTGTTGTAGGCAGCAGACTTGTTGCAAGTTTGAACAATGTAAGCTATGT
TTCCCCAAGTATTGACATTCTCCAAGCATACTACAACAGGAACATAAGTG
GTGTTTACACCGAAGATTTTCCATTGAATCCCCCAGTAATCTATGATTTC
ACTGGAAACTTGACTAATCTCAATACCCCTGTTGAGGAAGGGACGAGGGT
AATAGTGGTAAATTATGGGGAAGGAGTTGAGATGGTGCTGCAAGCAACCC
AGATGGGGGCTGGTGGAAGTCACCCAATCCATTTGCACGGTTCTAGCTTC
TATTGGGTGGGAACAGGTTTCGGAAATTTCAACAATAAGACAGACCCAAG
GACTTATAATCTGGTTGATCCACCACTCATTAACACTGTCCATGTTCCTG
GTAGAAGATGGGTTGCCATCAGATTTTTTGCTACCAATCCCGGAGTATGG
TTTATGCATTGCCATTTGGAGAGGCATAGTAGCTGGGGAATGGACACTGT
TCTCATCGTGAGGAATGGTAAAACCAAGAAAACAAGCATCCGCCCACCGC
CATCTACCATGCCTCGTTGTCCTGGAACCTAGATTTGGTGTGTCAAATAA
AAAGCTTGTGTGCTTAAGTTTGTGTTTTTTTCTTGGCTTGTTGTGTTATG
AATTTGTGGCTTTTTCTAATATTAAATGAATGTAAGATCTCATTATAATG
AATAAACAAATGTTTCTATAATCCATTGTGAATGTTTTGTTGGATAATGA
TAGATGTACTAAAGAGAAGACGAAGAAATGGAAAGATAATTGATAGTTGA
ATCATAATGATAG

### 10. Full Gene Sequence for the Trindican Cassette:

ACAACTTTTCTCTACCAATCAAATTTAAAAAATAGAAAAATGAAAATCGA
TGAATTGGATCACCACAATTTAGCCCAAAGAAAAACACAGTCAACCCCTC
TCACAGGGTAGGAATGATTTCGAGGTATAGATAGACATAGTAACGGGCAA
CTTTAACTATTGCTGCCTCGATTTGAGGAAAATATCAAATCCAAGACAAA
AATTTCAATTATACACTATGCCATACCATTATAAATATCCCCGTTCGCAA
TATCATCACCATTATTTGAATTTGCATTGCAACATTCGTCACCGTTAGTT
ATACCATCACCATCACTTAATTACTAAAATAATTATTGGTTTCTCAATAT
GAAAAAGCTCGAGTGCATTTTCTTTTGAATATCAACCGAAAAGAAAGGAA
AAACTAAAGATTTTGGAAGGTGACGGGGAAACCAAAAAGGAAATTTTGGG
CATTTTTAAAATGAGAAAGACGAATGTAATAACCCATTTTTCTTTCTTAC
TCTGACAACGCCACAGATGCTTTACATGCATCATGTGATCGTGGGGGACC
CGAAACTTGGCATACGGAAAGCACCAACGGCACAGCATTAAAAGAAATTG
TGTATAATGTTAAAAGACCATTAATTCAGTCTCATGGAAAATTTTAAACA
TCTTCCTGAACCTTTTCGTATTCGTGTTATTGAACCTGTTAAACGTACTA
CTCGTGCTTATCGTGAAGAAGCTATTATTAAATCTGGTATGAATCCTTTT
CTTCTTGATTCTGAAGATGTTTTTATTGATCTTCTTACTGATTCTGGTAC
TGGTGCTGTTACTCAATCTATGCAAGCTGCTATGATGCGTGGTGATGAAG
CTTATTCTGGTTCTCGTTCTTATTATGCTCTTGCTGAATCTGTTAAAAAT
ATTTTTGGTTATCAATATACTATTCCTACTCATCAAGGTCGTGGTGCTGA
ACAAATTTATATTCCTGTTCTTATTAAAAAACGTGAACAAGAAAAAGGTC
TTGATCGTTCTAAAATGGTTGCTTTTTCTAATTATTTTTTTGATACTACT
CAAGGTCATTCTCAAATTAATGGTTGTACTGTTCGTAATGTTTATATTAA
AGAAGCTTTTGATACTGGTGTTCGTTATGATTTTAAAGGTAATTTTGATC
TTGAAGGTCTTGAACGTGGTATTGAAGAAGTTGGTCCTAATAATGTTCCT
TATATTGTTGCTACTATTACTTCTAATTCTGCTGGTGGTCAACCTGTTTC
TCTTGCTAATCTTAAAGCTATGTATTCTATTGCTAAAAAATATGATATTC
CTGTTGTTATGGATTCTGCTCGTTTTGCTGAAAATGCTTATTTTATTAAA
CAACGTGAAGCTGAATATAAAGATTGGACTATTGAACAAATTACTCGTGA
AACTTATAAATATGCTGATATGCTTGCTATGTCTGCTAAAAAAGATGCTA
TGGTTCCTATGGGTGGTCTTCTTTGTATGAAAGATGATTCTTTTTTTGAT
GTTTATACTGAATGTCGTACTCTTTGTGTTGTTCAAGAAGGTTTTCCTAC
TTATGGTGGTCTTGAAGGTGGTGCTATGGAACGTCTTGCTGTTGGTCTTT
ATGATGGTATGAATCTTGATTGGCTTGCTTATCGTATTGCTCAAGTTCAA
TATCTTGTTGATGGTCTTGAAGAAATTGGTGTTGTTTGTCAACAAGCTGG
TGGTCATGCTGCTTTTGTTGATGCTGGTAAACTTCTTCCTCATATTCCTG
CTGATCAATTTCCTGCTCAAGCTCTTGCTTGTGAACTTTATAAAGTTGCT
GGTATTCGTGCTGTTGAAATTGGTTCTTTTCTTCTTGGTCGTGATCCTAA
AACTGGTAAACAACTTCCTTGTCCTGCTGAACTTCTTCGTCTTACTATTC
CTCGTGCTACTTATACTCAAACTCATATGGATTTTATTATTGAAGCTTTT
AAACATGTTAAAGAAAATGCTGCTAATATTAAAGGTCTTACTTTTACTTA
TGAACCTAAAGTTCTTCGTCATTTTACTGCTAAACTTAAAGAAGTTGGAA
GTGGAGAAGGAAGGGGATCTCTCACCTGCGGTGACGTCGAGGAGAACCCA
GGACCTATGGAACGTAAAGTTGGTATTATTGGTGCTGGTATTTCTGGTCT
TCTTGCTTGTAAACATGCTCTTTCTAAAGGTTTTCATCCTGTTGTTCTTG
AAGCTCAACCTGATATTGGTGGTGTTTGGGCTAATGCTCTTGAAACTACT
CGTCTTCAAACTCCTAAAGATTTTTATCAATTTTCTGATTTTCCTTGGCC
TTCTTCTGTTCAAGATATGCATCCTACTGGTGAACAAGTTATGGAATATA
TTCGTTCTTATGCTGATCATTTTGGTCTTCTTAAACATGTTCGTTTTAAT
ACTAAAGTTCTTTCTATTTCTTTTGAAGGTACTTCTGATCAAGAAATGGA
AGCTTGGTCTCATTGGAATGGTACTGGTGAAGGTTTTGCTAATAAAGGTA
AATGGACTCTTCTTCTTCAAGATGTTCCTACTCAATCTCAACAAGTTATG
GAATTTAATTTTGTTATTCTTGGTATTGGTAAATTTTCTGATCGTATGCG
TATGCCTGAATTTCCTGCTGGTGAAGGTCCTGAAGTTTTTGATGGTAAAG
TTATTCATTCTAAAGATTATTCTGAAAAATCTTATGGTGATGCTCGTCGT
CTTGTTAAAGGTAAACGTGTTGTTGTTGTTGGTTTTCAAAAATCTGCTAT
TGATATTGCTAATGAATGTTCTTCTGCTAATGGTAAAGAAGTTCCTTGTA
ATCTTATTTATCGTACTCCTCCTTGGAATATTCCTGATTTTTTTATTTGG
GGTCTTCCTCTTCCTTATCTTTATTTTAATCGTTTTTCTGAACTTCTTCT
TCATAAACCTGGTGAAGGTCTTTTTCTTTCTCTTCTTGCTACTATTCTTT
CTCCTCTTCGTTGGCTTATTGCTAAATTTGTTGAATCTAATATTAAATAT
CGTCATCCTCTTAAAAAATATGGTCTTGTTCCTGAACATGGTTTTGGTGA
AGCTATTTCTTCTTGTGTTCTTTCTGTTCTTCCTAAAGGTTTTTATGAAA
ATCTTGAAAAAGGTTCTATTCTTCTTAATAAAGCTGAAAAATTTCGTTTT
TGTAAAGAAGGTATTGTTATGGAAGGTAAACCTAAACCTCTTGAAGCTGA
TCTTGTTATTTTTGCTACTGGTTTTGAAGCTGATGAAAAACTTCGTGATA
TTTTTGCTTCTCCTAAATTTCAAGGTCATATTATGGGTTCTCCTCATTCT
ATTCTTCCTCTTTATCGTCAATGTATTCATCCTCAAATTCCTCAACTTGC
TATTATTGGTTATTCTGAATCTTTTGCTAATCTTTATACTTCTGAAATGC
GTTGTCGTTGGATTGTTGAACTTCTTGATGGTACTTTTAAACTTCCTGGT
TTTAAAGAAATGGAAAAAGAAACTGCTAATTGGAATAAATTTATTAAAGG
TAATGCTCCTAAACATTATCGTCGTTCTTGTATTGGTCAACTTCATATTT
GGTATAATGATCAACTTTGTAAAGATATGGGTTGGAATCCTCGTCGTAAA
AAAGGTTTTATTTCTGAATGGTTTCAACCTCATGGTCCTCTTGATTATAT
TGGTGGAAGTGGAGCTACTAACTTTCTCCTCCCAACAGGCTGGTGACGTC
GAGGAGAACCCAGGACCTATGGAATCTCCTGCTGCTCCTCCTACTACTGC
TCCTCCTCCTCATGTTATTATTATGCCTTCTGCTGGTATGGGTCATCTTA
TTCCTCTTGCTGAATTTGCTAAACGTCTTCTTCCTCGTTTTACTTTTACT
TTTGCTGTTCCTACTTCTGGTCCTCCTTCTTCTTCTCAACGTGATTTTCT
TTCTTCTCTTCCTGCTTCTATTGATACTTCTTTTCTTCCTGAAGTTGATC
TTTCTGATGCTCCTTCTGATGCTCAAATTGAAACTCTTATGTCTCTTATG
GTTGTTCGTTCTCTTCCTTCTCTTCGTGATCTTATTGCTTCTTATTCTGC
TTCTGGTCGTCGTGTTGCTGCTCTTGTTGTTGATCTTTTTGCTACTGATG
CTATTGATGTTGCTCTTGAACTTGGTATTCGTCCTTTTATTTTTTTTCCT
TCTACTGCTATGACTCTTTCTTTTTTTCTTCATCTTGAAAAACTTGATGA
AACTGTTTCTTGTGAATTTGCTGAACTTTCTGATCCTGTTCAAATTCCTG
GTTGTATTCCTGTTCATGGTAAAGATCTTATTGATCCTGTTCAAGATCGT
AAAAATGATGCTTATAAATGGCTTCTTCATCATTCTAAACGTTATAAACT
TGCTGAAGGTGTTATTGTTAATTCTTTTGAAGGTCTTGAAGGTGGTCCTA
TTCGTGAACTTCTTCATCCTGAACCTGGTAAACCTCGTGTTTATCCTGTT
GGTCCTCTTATTCAAGCTGGTTCTTGTGAAAAAGGTGCTGCTGCTCGTCC
TGAATGTCTTAAATGGCTTGATCAACAACCTCGTGGTTCTGTTCTTTTTG
TTAATTTTGGTTCTGGTGGTGTTCTTTCTACTGAACAACAAAATGAACTT
GCTGGTGTTCTTGCTCATTCTCAACAACGTTTTCTTTGGGTTGTTCGTCC
TCCTAATGATGGTATTGCTAATGCTACTTATTTTTCTGTTGATGGTGAAA
TTGATCCTCTTAAACTTCTTCCTGAAGGTTTTCTTGAACAAACTGCTGGT
CGTGGTCTTGTTCTTCCTATGTGGGCTCCTCAAATTGATGTTCTTTCTCA
TGAATCTACTGGTGGTTTTCTTACTCATTGTGGTTGGAATTCTACTCTTG
AATCTGTTTTTCATGGTGTTCCTCTTATTACTTGGCCTCTTTATGCTGAA
CAAAAAATGAATGCTGTTATGCTTACTGAAGGTCTTCGTGTTGGTCTTCG
TCCTTCTGTTGGTAAAGATGGTATTATTCGTGGTGATGAAATTGCTCGTG
TTATTGGTGAACTTATGGAAGGTGAAGAAGGTAAACGTATTCGTTCTAAA
ATGCAAGAACTTAAACGTGCTGCTTCTGCTGTTCTTTCTAAAGATGGTTC
TTCTACTCGTGCTCTTGAAGAAGTTGCTAAAATTTGGGAATCTAAAGTTT
AAATTTGGTGTGTCAAATAAAAAGCTTGTGTGCTTAAGTTTGTGTTTTTT
TCTTGGCTTGTTGTGTTATGAATTTGTGGCTTTTTCTAATATTAAATGAA
TGTAAGATCTCATTATAATGAATAAACAAATGTTTCTATAATCCATTGTG
AATGTTTTGTTGGATAATGATAGATGTACTAAAGAGAAGACGAAGAAATG
GAAAGATAATTGATAGTTGAATCATAATGATAG

### 11. Full Gene Sequence for the JOB1925 Marker:

TAATGATAGATGTACTAAAGAGAAGACGAAGAAATGGAAAGATAATTGAT
AGTTGAATCATAATGATAG

Although the encoded proteins are found in nature, the disclosed nucleic acid sequences are synthetic and non-naturally occurring. These sequences have been codon-optimized for expression in a plant host system and assembled into novel constructs with functional and structural distinctions that do not occur in nature. The resulting expression system yields enhanced performance and enables new utility—namely, the in planta biosynthesis of indigo pigment within plant vacuoles. The nucleic acid sequences encoding β-glucosidase, flavin monooxygenase, and indican synthase have been codon-optimized for expression in Gossypium hirsutum and do not occur naturally in this form.

Although the present invention has been described in considerable detail with reference to certain preferred versions thereof, other versions are possible. Therefore, the spirit and scope of the appended claims should not be limited to the description of the preferred versions contained herein. All features disclosed in this specification may be replaced by alternative features serving the same, equivalent or similar purpose, unless expressly stated otherwise. Thus, unless expressly stated otherwise, each feature disclosed is one example only of a generic series of equivalent or similar features.

1. A system comprising a genetically engineered fusion enzyme configured for in vivo biosynthesis of indigo pigment within plant cell vacuoles, the fusion enzyme comprising:
(a) a β-glucosidase domain derived from Polygonum tinctorium, configured to hydrolyze indican to form indoxyl;
(b) a laccase domain derived from Gossypium hirsutum, configured to facilitate oxidation of indoxyl to indigo;
(c) a flexible peptide linker comprising at least one GGGGS motif and at least one EAAAK motif, connecting the β-glucosidase domain and the laccase domain; and
(d) a vacuolar targeting signal that directs the fusion enzyme to the plant cell vacuoles.

2. The system of claim 1, wherein the β-glucosidase domain and laccase domain are expressed as a single polypeptide chain and functionally cooperate to convert indican to indigo within a plant cell vacuole.

3. The system of claim 1, wherein the vacuolar targeting signal is derived from a truncated CT10 peptide.

4. The system of claim 1, wherein the β-glucosidase domain lacks a chloroplast transit peptide and is codon-optimized for expression in a plant cell.

5. The system of claim 1, wherein the laccase domain comprises the full-length native sequence of GhLAC1 from Gossypium hirsutum.

6. The system of claim 1, further comprising a fluorescent protein tag selected from the group consisting of GFP, RFP, YFP, and mCherry, operably linked to the fusion enzyme for subcellular localization analysis.

7. The system of claim 1, wherein the flexible peptide linker consists of (GGGGS)₃ and (EAAAK)₁ motifs arranged to promote proper folding and activity of the β-glucosidase and laccase domains.

8. The system of claim 1, wherein the fusion enzyme is encoded by a recombinant expression vector comprising: (a) a promoter operable in plant fiber cells; (b) a nucleic acid sequence encoding the fusion enzyme; (c) a transcription terminator; and (d) a selectable marker.

9. The system of claim 8, wherein the promoter is selected from the group consisting of GhSCFP, E6, and any fiber-specific promoter active during cotton fiber elongation or secondary wall synthesis.

10. A method of producing indigo pigment within plant fiber cells, the method comprising:
(a) transforming a plant with the system of claim 18;
(b) cultivating the plant under suitable growth conditions;
(c) expressing the fusion enzyme in a fiber-specific manner; and
(d) enzymatically converting indican to indigo within the vacuoles of the fiber cells.

11. The method of claim 10, wherein the plant is a transgenic Gossypium hirsutum plant.

12. The method of claim 10, wherein the promoter is selected from the group consisting of GhSCFP, E6, and any promoter preferentially active during cotton fiber development.

13. The method of claim 10, wherein the indigo pigment is confined within the vacuolar lumen of the fiber cells.

14. The method of claim 10, wherein the presence of indigo is confirmed using an analytical technique selected from the group consisting of high-performance liquid chromatography (HPLC), liquid chromatography-mass spectrometry (LC-MS), and UV-visible spectroscopy.

15. The method of claim 10, wherein the indican substrate is synthesized in planta via expression of a tryptophanase, a flavin monooxygenase, and an indican synthase.

16. A transgenic plant comprising the system of claim 1, wherein the fusion enzyme is expressed in a tissue-specific manner in fiber cells and catalyzes the in vivo biosynthesis of indigo pigment within vacuoles.

17. A cotton fiber derived from the plant of claim 16, wherein the fiber comprises indigo pigment produced by the system of claim 1 and deposited intracellularly during fiber development.

18. The system of claim 8, the system further comprising a tricistronic expression cassette configured to convert tryptophan to indican in a plant cell, the tricistronic expression cassette comprising:
(a) a nucleic acid sequence encoding a tryptophanase (TnaA) codon-optimized for plant expression, and configured to convert tryptophan into indole;
(b) a nucleic acid sequence encoding a flavin-containing monooxygenase (PtFMO) derived from Persicaria tinctoria, configured to convert indole into indoxyl; and
(c) a nucleic acid sequence encoding an indican synthase (PtIGS) derived from Persicaria tinctoria, configured to glucosylate indoxyl into indican;
wherein the three coding sequences are separated by self-cleaving peptide sequences selected from the group consisting of T2A and P2A, and are expressed under the control of a fiber-specific promoter and a plant-compatible terminator.

19. The system of claim 18, wherein the tricistronic expression cassette further comprises a selectable genetic marker designated JOB1925, located downstream of the terminator, and configured for detection by PCR.

20. A method of producing indican within a plant cell, comprising transforming a plant with the system of claim 18 and expressing the cassette in a fiber-specific manner to synthesize indican from tryptophan in vivo.

21. A transgenic plant comprising the system of claim 18 and capable of producing indican within plant fiber cells.
